(19) United States
(12) Patent Application Publication
(10) Pub. No.: US 2005/0048474 A1
(43) Pub. Date: Mar. 3, 2005

(54) Process and apparatus for separating and recovering particles from a liquid sample

(76) Inventor: James Emanuel Amburgey JR., Lawrenceville, GA (US)

Correspondence Address: James E. Amburgey, 2291 Plantation Court, Lawrenceville, GA, 30044, US

(21) Appl. No.: 10/920,992

(22) Filed: Aug. 18, 2004

Related U.S. Application Data
(60) Provisional application No. 60/496,101, filed on Aug. 18, 2003.

(51) Int. Cl. C12Q 1/70; C12Q 1/04

(52) U.S. Cl. 435/5; 435/34

(57) ABSTRACT

The process and apparatus of this invention can be used to separate and recover particles from a liquid sample. The preferred embodiment of this invention is to separate and recover microorganisms, particularly Cryptosporidium oocysts, from a variety of environmental sources. The process consists of applying a municipal drinking water coagulation scheme to an environmental water sample, and the aforementioned water sample is then filtered through an apparatus containing a granular filter material. The analyte is then recovered by agitating the filter vigorously (with a dispersing agent inside) and removing the eluent from the filtration apparatus. The eluent can then by aliquotted and assayed by any means available. The floc resulting from the coagulant addition can typically be dissolved at low pH. The filter apparatus is simply a container that allows the passage of water through a granular material while retaining the granular material via a mesh, screen, or similar material.

### CROSS-REFERENCE TO RELATED APPLICATIONS

This application claims priority to the provisional patent application No. 60/496,101, filed Aug. 18, 2003, entitled “Method and Apparatus for Separating Analyte from a Sample.”

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

This new extraction process and apparatus are primarily intended to be used to separate and recover microbial contaminants (e.g., bacteria, viruses, and/or protozoa) from water samples. The water samples may be drawn from various stages of treatment of municipal or industrial water and wastewater treatment facilities, or the water samples can drawn from the natural environment (e.g., lakes, rivers, or oceans) or the laboratory setting.

The predominant reason for a new extraction process and apparatus is the high cost and often low and/or highly variable recovery efficiencies of the current technologies. The Gelman Envirochek filters commonly used in United States Environmental Protection Agency (USEPA) Method 1622/1623 can cost nearly $100 each and cannot be reused. The recovery of Cryptosporidium oocysts from environmental sources and drinking water supplies has already been studied extensively. DiGiorgio et al (2002) reported Cryptosporidium recoveries ranging from 36 to 75% for surface water samples. McCuin and Clancy (2003) reported mean Cryptosporidium recoveries of 48.4% for filtered tap water samples, 19.5 to 54.5% for raw source water samples, and 2.1 to 36.5% recovery for matrix spikes. The method and apparatus described by this patent were invented with Cryptosporidium analysis in mind, but this new technology can also be applied to other tasks where it is desirable to concentrate the particles found in liquid sample.

One potential new application for this technology is for responding to the threat of a terrorist attack on a potable water supply with a biological agent. The traditional approach to sample extraction methods has been to tailor the method to only recover a single or narrow range of biological target(s). There is no need for a sample concentrate to contain bacteria, fungi, algae, and protozoa if only the viruses in the sample concentrate will be assayed. Furthermore, extraction methods are often tailored to recover only one or two target organisms and may not even capture smaller organisms of the same family. This strategy is fine as long as the investigator knows exactly what he is looking for and selects the appropriate extraction method. The problem is that in the event of a terrorist attack the analytical staff may not know anything about the biological agent that is sought. Since it is not practical to go into the field and collect water samples from multiple locations with each of 4 or more types of filters each with its own pretreatment requirements and eluting solutions, a universal extraction method to concentrate all potential biological targets in an efficient, economical, and timely manner would be beneficial.

### BRIEF SUMMARY OF THE INVENTION

This invention is based on intuitive application of the principles of drinking water treatment to the analysis of microbial contaminants in water samples. Drinking water treatment typically relies on the combined processes of coagulation and granular filtration to remove microbial contaminants from waters intended for human consumption (Amirtharajah and O'Melia, 1990; Cleasby and Logsdon, 1999). Following the removal of coagulated particles and microbial contaminants, filters become clogged with deposits and must be cleaned via backwashing (or forcing a stream of water upward through the filter to remove the deposited materials). This invention applies coagulation and filtration technologies to the new purpose of microbial analysis. Instead of using water treatment technologies to produce clean water, these technologies are applied to separate microorganisms from the original water samples for later recovery and concentration into a smaller volume of water for subsequent analysis. The clean water is simply discarded as a byproduct of this process. The recovery process is essentially a modified backwash procedure whereby additional measures are taken to improve recovery during the wash step. The additional measures can include physical agitation and chemical addition to the washwater stream to promote detachment and prevent reattachment of the target particles. The new apparatus is a strategically designed system to accommodate and control the aforementioned processes in a cost-effective manner while achieving efficient recoveries of particles into more manageable volumes of water. This low-technology solution is quite inexpensive and highly effective at recovering particles.

### BRIEF DESCRIPTION OF THE SEVERAL VIEWS OF THE DRAWINGS

Not Applicable

### DETAILED DESCRIPTION OF THE INVENTION

Granular media filtration has been used for more than 100 years to efficiently remove pathogens from drinking water, but this technology had not previously been successfully adapted to microbial sampling methodologies. Physicochemical treatment of drinking water usually involves coagulation (e.g., adding aluminum sulfate or alum), flocculation (or gentle mixing), sedimentation, and finally granular media filtration. The first three steps of drinking water treatment (i.e., coagulation, flocculation, and sedimentation) have already been incorporated into Standard Method #9510 D for concentrating viruses in water samples (APHA, 1995). There is an option in Standard Method #9510 D to replace the sedimentation step (i.e., centrifugation) with a filtration step, but a standard flat membrane filter is listed as the only option. This new process and apparatus are centered around the use of a granular media filter with a different coagulation scheme and appropriate recovery techniques for a granular media filter approach. While there is certainly some overlap in the technologies applied in this invention with both water treatment practice and an existing microbial method, this invention is the first application of a granular media filtration based approach to recovering microorganisms from water samples. Despite some conceptual similarities, the Standard Method #9510 D is very different from this new process with the two methods sharing none of the same procedural steps.

Karanis and Kimura (2002) reported Cryptosporidium recovery results with three coagulation-flocculation-sedimentation methods, which utilized different coagulants. However, Karanis and Kimura (2002) did not use any type of filtration process to separate the Cryptosporidium oocysts from the water samples preferring to leave samples sitting overnight for sedimentation to slowly separate the oocysts. After 24 hours, Karanis and Kimura (2002) carefully removed the supernatant without disturbing the sediment by using a vacuum pump. Zanelli et al (2000) used methods similar to Karanis and Kimura (2002) also using sedimentation instead of filtration to concentrate the Cryptosporidium oocysts. The new process and apparatus described in this patent are intended to be used with a granular media filter instead of a sedimentation step. The new process and apparatus uses a different coagulant at a different dosage and at a different final pH to ensure excellent removal via the granular media filter. The new process is partially based on the conditions shown to be effective in water treatment practice instead of those conditions used in previous analytical methods for selected microorganisms. In short, the new method and apparatus differ in many important ways from any previous analytical methods and apparatuses to accommodate the granular media filter approach in an effective manner.

The power of granular media filters is that they can efficiently remove particles of a very broad range of sizes without rapidly clogging like membrane filters because granular filters collect particles throughout the depth of the media. For granular filters, the removal process occurs by particles attaching to the media surface and later to other particles already removed. Membrane filters rely on very tiny opening to allow water to pass while retaining the particles of interest, but when the holes become clogged the membrane rapidly ceases to process more water. Granular media filters usually have channels for the water to flow through that are 0.155 times the diameter of the media (Stevenson, 1997). A filter with 0.5-mm diameter sand media would have channels approximately 77.5 microns in diameter, which allows for a significant number of 1-micron diameter bacteria or 5-micron diameter Cryptosporidium oocysts to collect before the filter begins to clog. Furthermore, the granular media filter does not compress the removed material into a solid cake on the surface of a stationary flat membrane from which removal can be difficult and the incorporation of the particles of interest into larger agglomerates can make them inaccessible for later analytical techniques. Due to their many inches of depth, granular media filters have adequate surface area to prevent the formation of surface cakes (unlike flat membrane filters that have little or no useable depth), and the granular media is mobile when the filter is shaken to allow more effective removal and assist in breaking up any loosely agglomerated materials. Typical granular media filters used by water treatment utilities contain 24 to 36 inches of media, but these filters are designed to operate for 24 to 144 hrs without backwashing while the “active layer” of the filter moves progressively deeper as the upper regions of the filter begin to clog. Using a shallower depth of media is possible with 10 L samples that can be filtered in roughly 30 minutes. Filter effluent turbidity may be a good indicator of the performance of the filter in removing target biological agents and can be used to determine an appropriate depth of media to use in the filters.

The issue of proper coagulation is not one that can be ignored if high removals of any type of particle are sought with a granular media filter. If the coagulation conditions are not appropriate, then significant quantities of particles will pass directly through a granular filter that has pores larger than the particles being collected in many cases. Water treatment plants typically perform tests on a regular basis to ensure proper coagulation is being achieved, and the coagulant dose can change with water quality or even temperature. Extensive jar tests to determine the optimum coagulant dose and coagulated water pH are not feasible for a microbial testing method, but they are not necessary. Water treatment plants treat large quantities of water on a continual basis, and a small decrease in coagulant dose can mean saving money in chemical costs and sludge disposal fees. This is why water operators strive to use the minimum dose of coagulant possible, which frequently corresponds to a regime of coagulation known as “charge neutralization.” With charge neutralization, a coagulant dose that is too low leaves a negative surface charge on the particles that may preclude removal within the filter, but a coagulant dose that is too high can result in charge reversal to a positive surface charge that can also preclude removal in the filter as like charges (both positive or both negative) repel. Fortunately, a coagulation regime exists that tends to be rather stable regardless of the particle concentration, which is commonly called “sweep coagulation.” Most water treatment plants do not use sweep coagulation because of the significantly higher chemical dosages required and correspondingly higher sludge disposal requirements. However, sweep coagulation is a robust process that can perform well with very low particle concentrations (e.g., a tap water sample) or very high particle concentrations (e.g., a raw water sample). It is theorized that the floc particles formed during sweep coagulation are not positively charged such that they must combine in an exact ratio to neutralize the particle's original surface charge, but the sweep floc is itself of a neutral surface charge and simply masks that original surface of the particles as it coats or engulfs them. The floc is fragile and easily broken apart when desired. Excess coagulant will form excess precipitant causing the filter to clog prematurely or deteriorated particle removal during the later portion of a filtered sample. Thus, the coagulation conditions are extremely important to the success of the process.

Water treatment is a very flexible and adaptable process. There are multiple coagulants that can be used (e.g., aluminum sulfate, aluminum chloride, ferric sulfate, ferric chloride, polyaluminum chloride, polyferric sulfate, chitosan, and cationic polymers) with many coagulants requiring a specific pH range to achieve excellent results. Water treatment processes can often be improved by adding additional chemicals to aid in the process (i.e., coagulant aids or flocculent aids), which include: anionic polymer, nonionic polymer, cationic polymers, clay, activated silica, sand, and even dissolved calcium ions. There are multiple existing filter medias that can be used as well. Currently used filter medias include: sand, anthracite coal, granular activated carbon, garnet, ceramic media, and even some glass and plastic medias. The method and apparatus described in this patent are also expected to be very flexible in practice with some select substitutions and changes making little difference in method performance.

Existing microbial analytical methods also tend to be very flexible. For example, a solution containing a specified concentrations of a certain number of chemicals might be used to recover microorganisms attached to the surface of a membrane filter. One constituent of the aforementioned solution could be a surface-active agent (surfactant), or “soap” as they are commonly called. However, there are probably dozens of surfactants that would perform a similar function in the solution besides the one that is used most frequently. Modest increases or decreases in the concentration of a constituent would also be unlikely to significantly impact the overall method performance.

An innovative dispersant technology (McCuin et al, 2000) that has improved Cryptosporidium recovery in large volume samples analyzed by the membrane filters used in USEPA Method 1622/1623 may be used to improve analyte recovery from the granular filter apparatus described herein. Dispersing agents have not traditionally been taken advantage of in published extraction methodologies. A dispersant works by attaching to the surface of particles and/or surfaces thereby increasing the magnitude of their original negative or neutral surface charges to promote electrostatic repulsion and deter particles attaching to other particles or surfaces. Dispersants could also be used in place of (or in addition to) the standard eluting solutions in this technique. Sodium metaphosphate is common dispersing agent that is often used at concentrations of 0.5 to 5 g/L. Some other common dispersants are pyrophosphate, polyphosphates, and silicates. Each dispersant has slightly different properties, but each performs a similar function in a solution. After a solution for recovering microbes is applied to a filter, there is typically some form of physical agitation that is intended to help detach particles and break agglomerated materials. Some common forms of physical agitation include wrist-action shaking, vortex mixing, shaking by hand, and sonication. Many different forms of physical agitation or combinations thereof could achieve similar results, which again demonstrates the inherent flexibility of this type of analytical method.

This invention will be further illustrated by the following example:

After filling a carboy having a drain valve at the bottom with 10 L of tap water, 30,000 4.5-micron diameter fluorescent microspheres (or 70,000 Cryptosporidium oocysts) were added and mixed by repeated inversions and shaking for 15 seconds. Next, 3.5 mL of a 10% by weight ferric chloride solution was added and mixed by inversions. Then, 6.4 mL of 1N sodium hydroxide was added and mixed by inversions to achieve a target pH of 7.4 (+/−0.3). The solution was then allowed to stand undisturbed for 10 minutes before the carboy was swirled for 5 seconds just prior to filtration to resuspend any settled materials.

A 2.0-inch diameter, 8 inch long granular media filter apparatus containing 5 inches of pre-cleaned sand (cleaned by repeated air-scour backwashing and sonication steps) or crushed recycled glass (cleaned only by repeated air-scour backwashing) was filled with water (at a rate of 500 mL/min) and then gently fluidized at a rate of 1700 mL/min for 1 minute to ready the filter for use. The backwash effluent tubing was removed and replaced with a section of filter influent tubing to connect the filter to the carboy containing the 10 L sample. The filter was tapped gently on the side to compact the media in the bed. The filter influent line was closed with a clamp and the backwash influent tubing was replaced with a filter effluent drain line with a flow controller attached. The flow controller was a tiny orifice fashioned from a ⅛ inch to 1/16 inch tubing reduction connector that was drilled to a slightly larger size to achieve the desired initial flow rate of 330 mL/min (or 4 gallons per min per sq. ft.). After removing the clamp, the sample flowed at target rate 330 mL/min initially and gradually decreased to 240 mL/min as the water level in the carboy dropped and decreased the hydraulic head of the system. When approximately 1 L was remaining in the carboy, the carboy was swirled gently to resuspend any settled materials, and the carboy was tilted to ensure that all of the sample was transferred from the carboy to the filter. The filtration process takes approximately 33 minutes to complete under these conditions, but this process is completely scalable with a potential trade-off between faster processing times and larger recovery volumes.

The filter was drained completely before reconnecting the backwash pump tubing. The filter was then refilled to 1 inch above top of media with a solution of 0.5% (5 g/L) Polyphosphate at a rate of 300 mL/min. The backwash effluent line at the top of the filter was then clamped off, and the backwash influent line was then removed and replaced by a clamped off section of tubing. The filter was then shaken by hand holding on to both ends for 30 seconds. The backwash tubing was then reconnected and the filter was backwashed at 500 mL/min until backwash effluent was almost clear (˜200 mL) with the backwash effluent stream being collected in a 200 mL conical centrifuge tube. A second sequence of shaking and backwashing was followed with the second sample being collected in a separate 200 mL tube. The recovery efficiency of the microspheres was then determined by assaying 1 mL of each sample by passing it through a polycarbonate track-etched filter with 3-micron diameter pores that was mounted on a microscope slide and counted at 100× total magnification with an epifluorescent microscope. Cryptosporidium analysis required immunofluorescent staining and microscopic observations at 250× total magnification.

These recovery values are reported in Table 1 for varying depths of sand as the filter media. The mean recovery of the aforementioned experiments was approximately 90% with the second backwash sample contributing approximately 10% to the total recovery. One experiment conducted to date with crushed recycled glass filter media yielded approximately 95% recovery with a single backwash and 100% recovery with the second backwash. The crushed recycled glass filter media seems much easier to clean effectively prior to use and will replace the sand media in future experiments. Any further sample concentration will likely necessitate the dissolution of the floc created by adding the ferric chloride coagulant, which can be achieved by adding 0.5 grams of oxalic acid to the centrifuge tube (to lower the pH below 2.0) and allowing the sample to sit for 10 minutes. Centrifugation at 3000×g for 20 minutes and aspiration was sufficient to concentrate the particles of interest down to approximately 10 mL. A wide variety of additional steps are applicable on the analyte chosen and the type of assay.

TABLE 1
Total recovery with varying depths of sand in a
granular media filtration apparatus

| | Depth of | |
|---|---|---|
| | Sand (inches) | % Recovery |
| Microspheres Exp. #1 | 5.8 | 97.8 |
| Microspheres Exp. #2 | 2 | 66.6 |
| Microspheres Exp. #3 | 4 | 100 |
| Microspheres Exp. #4 | 4.3 | 98.4 |
| Microspheres Exp. #5 | 4 | 81.1 |
| Microspheres Exp. #6 | 6 | 99.7 |
| Microspheres Exp. #7 | 5.1 | 88.2 |
| Cryptosporidium Exp. #7 | 5.1 | 91.6 |
| Mean | | 90.4 |
| Standard Deviation | | 11.7 |

The experimental apparatus is a relatively simple design four necessary components. The first component was a 10 L carboy with screw cap top and a spout at the bottom to hold the water sample. The second component was the filtration apparatus, which was fashioned from a 8-inch long (2.0-inch inner diameter) section of acrylic pipe. Each end of the filter apparatus was sealed with a #11 rubber stopper with one ½-inch diameter hole in the center. The hole in each stopper was filled with hose barbed reducing fittings of ⅝-inch by ¼-inch diameter with the larger openings inside the rubber stoppers. The lower rubber stopper was covered with a plastic mesh screen with holes small enough to retain 0.5 mm granular filter materials (e.g., sand, coal, or recycled crushed glass). The filter apparatus was filled with the third component of the experimental apparatus, which was approximately 5 inches of sand (effective size (E.S.) of 0.6 mm and a uniformity coefficient (U.C.) of 1.4) or crushed recycled glass (E.S.=0.5 mm and U.C.=1.4) leaving at least 2 inches of empty space above the filter media (for expansion of the media during backwashing during which air bubbles can escape from the media). The final component of the experimental setup was a small (˜1/16 inch diameter) plastic orifice at the end of a section of silicone tubing that was small enough to restrict the flow of water through the orifice to the desired level. The flow through the system can be controlled by the distance between the water level in the carboy and the point of discharge from the restriction orifice as well as the size of the restriction orifice. The carboy was connected to the filter apparatus filled with granular media via a section of ¼-inch inner diameter silicone tubing. The carboy was positioned above the filter apparatus and the restriction orifice to facilitate gravity flow of the water sample through the filter. The section of tubing leaving the filter apparatus and ending with the restriction orifice emptied into a second carboy. The cap on the carboy containing the original 10 L sample must be loosened to facilitate flow from the carboy. A ring stand and clamp were used to secure the filtration apparatus in place, and the original carboy was placed on top of an existing shelf above the benchtop. The second carboy was placed on the floor in the laboratory to collect the filtrate. A digital peristaltic pump was used to force the recovery solution up through the filter during the recovery step, and the recovered samples were collected from the end of a short section of ¼-inch inner diameter silicone tubing in a 200 mL conical bottom polypropylene centrifuge tube.

### References Cited

U.S. Patent Documents

None

### Other References

APHA, AWWA, and WEF. (1995). Standard Methods for the Examination of Water and Wastewater, 19^{th} ed. APHA, Washington, D.C. ISBN: 0875532233

Amirtharajah, A. and O'Melia, C. R. (1990). Coagulation Processes: Destabilization, Mixing, and Flocculation. In Water Quality and Treatment 4^{th} ed., Pontius, F. W., ed. McGraw-Hill Inc., New York. ISBN: 0070015406

Cleasby, J. L., and G. S. Logsdon. (1999). Granular Bed and Precoat Filtration. In Water Quality and Treatment, 5^{th} ed. McGraw-Hill, New York. ISBN: 0070016593

DiGiorgio, C. L., Gonzalez, D. A., and Huitt, C. C. (2002). Cryptosporidium and Giardia Recoveries in Natural Waters by Using Environmental Protection Agency Methods 1623. Applied and Environmental Microbiology, 68(12): 5952-5955

Karanis, P. and Kimura, A. (2002). Evaluation of Three Flocculation Methods for the Purification of Cryptosporidium Parvum oocysts from Water Samples. Letters in Applied Microbiology, 34 (2002): 444-449.

McCuin, R. M., Hargy, T. M., Amburgey, J. E., and J. L. Clancy. (2001). Improving Methods for Isolation of Cryptosporidium Oocysts and Giardia Cysts from Source and Finished Water. CD-ROM Proceedings American Water Works Association Water Quality Technology Conference, Nashville, Tenn.

McCuin, R. M., and Clancy, J. L. (2003). Modifications to United States Environmental Protection Agency Methods 1622 and 1623 for Detection of Cryptosporidium Oocysts and Giardia Cysts in Water. Applied and Environmental Microbiology, 69(1): 267-274.

Stevenson, D. G. 1997. Water Treatment Unit Processes. Imperial College Press, London, UK. ISBN: 1860940749.

Zanelli, F., Compagnon, B., Joret, J. C., and deRoubin, M. R. (2000). Enumeration of Cryptosporidium Oocysts from Surface Water Concentrates by Laser-scanning Cytometry. Water Science and Technology, 41(7): 197-202.

1. A process for separation and recovery of particles from a liquid sample successively subjected to the following steps (a) to (c):
step (a) in which said liquid sample is mixed with one or more materials to promote agglomeration of said particles
step (b) in which the suspension delivered from step (a) is passed through a filtration apparatus to separate a portion of the said particles from the said liquid sample
step (c) in which said particles retained by the said filtration apparatus in step (b) are subjected to a treatment to recover the said particles from the said filtration apparatus.

2. The separation and recovery process of claim 1, wherein the liquid sample for step (a) is a water sample.

3. The separation and recovery process of claim 1, wherein the said material(s) to promote agglomeration for step (a) comprise at least one member selected from the group consisting of polyvalent metal salts, inorganic polymeric compounds containing one or more types of polyvalent metal ions, polymeric organic compounds, fine particulate materials, acids, and bases.

4. The separation and recovery process of claim 1, wherein the said particles in step (a) comprise at least one member selected from the group consisting of viruses, bacteria, and protozoans.

5. The separation and recovery process of claim 1, wherein the said particles in step (a) comprise one or more types of Cryptosporidium oocysts.

6. The separation and recovery process of claim 1, wherein the said filtration apparatus in step (b) comprises a granular filtration material.

7. The separation and recovery process of claim 1, wherein the said filtration apparatus in step (b) comprises at least one member selected from the group consisting of sand, coal, ceramic material, glass, and plastic.

8. The separation and recovery process of claim 1, wherein the said filtration apparatus in step (b) comprises at least one member selected from the group consisting of crushed glass, recycled crushed glass, positively charged ceramic material, and uncharged ceramic material.

9. The separation and recovery process of claim 1, wherein the said filtration apparatus in step (b) comprises both a granular filtration material and a membrane filtration material.

10. The separation and recovery process of claim 1, wherein the treatment to recover the said particles from the said filtration apparatus in step (c) comprises one or more elution procedures.

11. The separation and recovery process of claim 1, wherein the treatment to recover the said particles from the said filtration apparatus in step (c) comprises one or more types of physical agitation.

12. The separation and recovery process of claim 1, wherein the treatment to recover the said particles from the said filtration apparatus in step (c) comprises one or more wash procedures whereby a liquid solution is forced through the said filtration material.

13. The separation and recovery process of claim 1, wherein the treatment to recover the said particles from the said filtration apparatus in step (c) comprises the application of a solution containing at least one member selected from the group consisting of pyrophosphates, polyphosphates, silicates, and surface active agents.

14. A process as claimed in claim 1, further comprising the application of an acid or base to dissolve some portion of the material recovered from the said filtration apparatus.

15. A process as claimed in claim 1, further comprising conducting an assay to determine the presence, identity, or number of said particles recovered from the said filtration apparatus.

16. A process as claimed in claim 1, further comprising at least one member selected from the group consisting of centrifugation, gradient separation, immunomagnetic separation, fluorescent staining, microscopy, cytometry, extraction of genetic material, and digestion.

17. Apparatus for separating and recovering particles from a liquid sample, comprising:
(a) a reservoir for holding a liquid sample containing particles;
(b) porous material(s) for use as filter media;
(c) a housing for retaining said filter media while allowing said liquid to pass through said filter media; and
(d) a means of introducing said liquid sample containing particles into said housing containing said filter media over a period of time.

18. The apparatus of claim 17, wherein said porous materials comprise at least one member selected from the group consisting of sand, coal, ceramic material, glass, plastic, and polymeric membrane material.

19. The apparatus of claim 17, wherein said housing for retaining said filter media comprises at least one member selected from the group consisting of glass, clear plastic, colored plastic, wire mesh, plastic mesh, porous plate, porous stone, porous plastic, and porous plastic aggregate material.

20. The separation and recovery process of claim 17, wherein the said means of introducing said liquid sample into said housing containing said filter media comprise at least one member selected from the group consisting of gravity induced flow, pressure induced flow, negative pressure induced flow, pump flow, tubing, piping, valves, and flow-restriction orifices.
